(19) United States
(12) Patent Application Publication
(10) Pub. No.: US 2024/0325685 A1
(43) Pub. Date: Oct. 3, 2024

(54) Urinary Catheters and Methods

(71) Applicant: C.R. BARD, INC, Franklin Lakes, NJ (US)

(72) Inventors: Kyle Daw, Smyma, GA (US); Ronald Legaspi, Alpharetta, GA (US)

(21) Appl. No.: 18/580,449

(22) PCT Filed: Jun. 29, 2022
(86) PCT No.: PCT/US2022/035565

§ 371 Date: Jan. 19, 2024

Related U.S. Application Data
(60) Provisional application No. 63/223,503, filed on Jul. 19, 2021.

(51) Int. Cl. A61M 25/00

(52) U.S. Cl. CPC A61M 25/0017; A61M 25/0045; A61M 2025/0046

(57) ABSTRACT

Disclosed herein are urinary catheters and methods for manufacture and use. The urinary catheter can include a catheter tube, a lubricious coating over a surface of at least a distal portion of the catheter tube, and a funnel coupled to a proximal portion of the catheter tube. The catheter tube can be of a polymeric material. The lubricious coating can include a hydrophilic coating over the surface of the catheter tube and lubricant over the hydrophilic coating. The funnel can include a funnel opening in a proximal end of the funnel for voiding urine. In an example of a method of a urinary catheter, the method can include a method of making or using the urinary catheter.

### PRIORITY

This application claims the benefit of priority to U.S. Provisional Patent Application No. 63/223,503, filed Jul. 19, 2021, which is incorporated by reference in its entirety into this application.

### BACKGROUND

Urinary catheter users or their caregivers have a limited amount of time for catheterization upon opening a urinary-catheter package including a prewetted hydrophilic coating-coated catheter. This is because water found in the hydrophilic coating begins to rapidly evaporate upon opening the urinary-catheter package. And once the water begins to evaporate, the coating starts to become tacky, which greatly increases the difficulty of the catheterization. What is needed is a urinary catheter that mitigates such water evaporation and resulting tackiness, thereby giving urinary catheter users or their caregivers adequate time for successful urethral insertion.

Disclosed herein are urinary catheters and methods that address the foregoing.

### SUMMARY

Disclosed herein is a urinary catheter including, in some embodiments, a catheter tube, a lubricious coating over a surface of at least a distal portion of the catheter tube, and a funnel coupled to a proximal portion of the catheter tube. The catheter tube is of a polymeric material. The lubricious coating includes a hydrophilic coating over the surface of the catheter tube and lubricant over the hydrophilic coating. Water of the lubricant is at least partially absorbed by the hydrophilic coating. The funnel includes a funnel opening in a proximal end of the funnel for voiding urine.

In some embodiments, the hydrophilic coating is a polyvinylpyrrolidone- or hyaluronic acid-based coating.

In some embodiments, the lubricant is a combination of glycerin, water, glyceryl acrylate/acrylic acid copolymer, and propylene glycol.

In some embodiments, the glycerin, glyceryl acrylate/acrylic acid copolymer, and propylene glycol limit evaporation of any water from the lubricious coating.

In some embodiments, the polymeric material is thermoplastic polyurethane (“TPU”), silicone, polyvinyl chloride, or a rubber.

In some embodiments, the surface of the catheter tube is a plasma-treated surface with a surface energy that promotes adhesion of the hydrophilic coating.

Also disclosed herein is a method of making a urinary catheter. The method includes, in some embodiments, a hydrophilic-coating applying step and a lubricant-dipping step. The hydrophilic-coating applying step includes applying a hydrophilic coating over a surface of a catheter tube of the urinary catheter. The lubricant-dipping step includes dipping at least a distal portion of the catheter tube into a reservoir of a lubricant, thereby disposing the lubricant over the hydrophilic coating. Water of the lubricant is at least partially absorbed by the hydrophilic coating.

In some embodiments, the lubricant-dipping step includes immersing the catheter tube in the lubricant with an immersion speed from about 0.15 in/s to about 0.50 in/s.

In some embodiments, the lubricant-dipping step includes immersing the catheter tube in the lubricant with an immersion speed of about 0.33 in/s.

In some embodiments, the lubricant-dipping step includes withdrawing the catheter tube from the lubricant with a withdrawal speed from about 0.05 in/s to about 0.20 in/s.

In some embodiments, the lubricant-dipping step includes withdrawing the catheter tube from the lubricant with a withdrawal speed of about 0.12 in/s.

In some embodiments, the lubricant-dipping step includes substantially no dwell time in the lubricant.

In some embodiments, the lubricant-dipping step includes a single dip in the lubricant.

In some embodiments, the lubricant is a combination of glycerin, water, glyceryl acrylate/acrylic acid copolymer, and propylene glycol.

In some embodiments, the glycerin, glyceryl acrylate/acrylic acid copolymer, and propylene glycol limit evaporation of any water from the lubricious coating.

In some embodiments, the method further includes a catheter-packaging step.

The catheter-packaging step includes packaging the urinary catheter without air drying the urinary catheter after the lubricant-dipping step.

In some embodiments, the method further includes a surface-treating step. The surface-treating step includes treating at least the distal portion of the catheter tube with a plasma treatment to modify the surface of the catheter tube before the hydrophilic-coating applying step.

In some embodiments, the catheter tube is of a polymeric material selected from thermoplastic polyurethane (“TPU”), silicone, polyvinyl chloride, and a rubber.

In some embodiments, the hydrophilic coating is a polyvinylpyrrolidone- or hyaluronic acid-based coating.

Also disclosed herein is a method of using a urinary catheter. The method includes, in some embodiments, a catheter package-obtaining step, an unpackaging step, a catheter-inserting step, and a urine-voiding step. The catheter package-obtaining step includes obtaining a packaged urinary catheter. The packaged urinary catheter includes a urinary catheter disposed in an outer packaging. A catheter tube of the urinary catheter includes a lubricious coating of a hydrophilic coating over a surface of at least a distal portion of the catheter tube and a lubricant over the hydrophilic coating. The unpackaging step includes removing the urinary catheter from the outer packaging by a funnel of the urinary catheter. The catheter-inserting step includes inserting the catheter tube into a urethra. The lubricious coating provides a user or his or her caregiver adequate time for successful urethral insertion of the catheter tube by limiting evaporation of any water from the lubricious coating form the unpackaging step through the catheter-inserting step that would otherwise cause tackiness. The urine-voiding step voiding urine from a bladder.

In some embodiments, the method further includes a catheter-withdrawing step. The catheter-withdrawing step includes withdrawing the catheter tube from the urethra after the urine-voiding step.

In some embodiments, the method further includes a repackaging step and a resealing step. The repackaging step includes placing the urinary catheter in the outer packaging. The resealing step includes resealing the urinary catheter in the outer packaging, thereby preventing residual urine leakage from the urinary catheter.

In some embodiments, the method further includes a disposal step. The disposal step includes disposing of the urinary catheter.

These and other features of the concepts provided herein will become more apparent to those of skill in the art in view of the accompanying drawings and following description, which describe particular embodiments of such concepts in greater detail.

### DRAWINGS

FIG. 1 illustrates a male urinary catheter in accordance with some embodiments.

FIG. 2 illustrates a female urinary catheter in accordance with some embodiments.

FIG. 3 illustrates a cross section of the male or female urinary catheter in accordance with some embodiments.

FIG. 4 illustrates a method of using the male urinary catheter in accordance with some embodiments.

FIG. 5 illustrates a method of using the female urinary catheter in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient or user. Likewise, a “proximal length” of, for example, the catheter includes a length of the catheter intended to be near the clinician when the catheter is used on the patient or user. A “proximal end” of, for example, the catheter includes an end of the catheter intended to be near the clinician when the catheter is used on the patient or user. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient or user when the catheter is used on the patient or user. Likewise, a “distal length” of, for example, the catheter includes a length of the catheter intended to be near or in the patient or user when the catheter is used on the patient or user. A “distal end” of, for example, the catheter includes an end of the catheter intended to be near or in the patient or user when the catheter is used on the patient or user. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, urinary catheter users or their caregivers have a limited amount of time for catheterization upon opening a urinary-catheter package including a prewetted hydrophilic coating-coated catheter. This is because water found in the hydrophilic coating begins to rapidly evaporate upon opening the urinary-catheter package. And once the water begins to evaporate, the coating starts to become tacky, which greatly increases the difficulty of the catheterization. What is needed is a urinary catheter that mitigates such water evaporation and resulting tackiness, thereby giving urinary catheter users or their caregivers adequate time for successful urethral insertion.

Disclosed herein are urinary catheters and methods that address the foregoing.

### Urinary Catheters

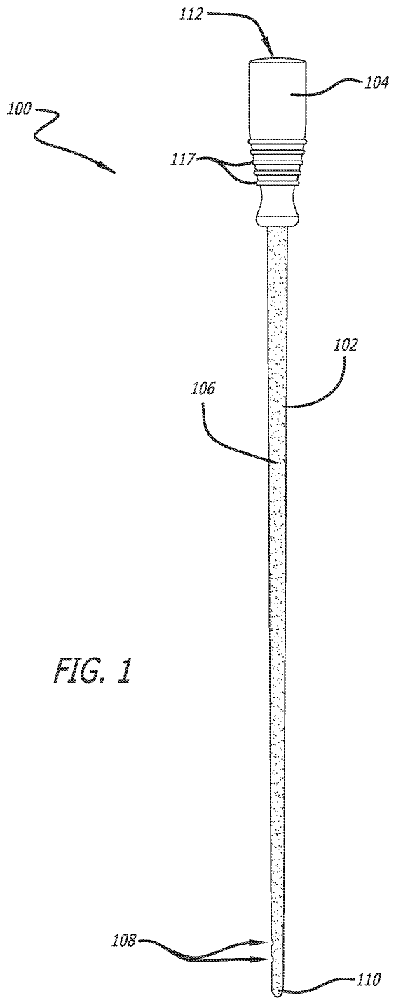

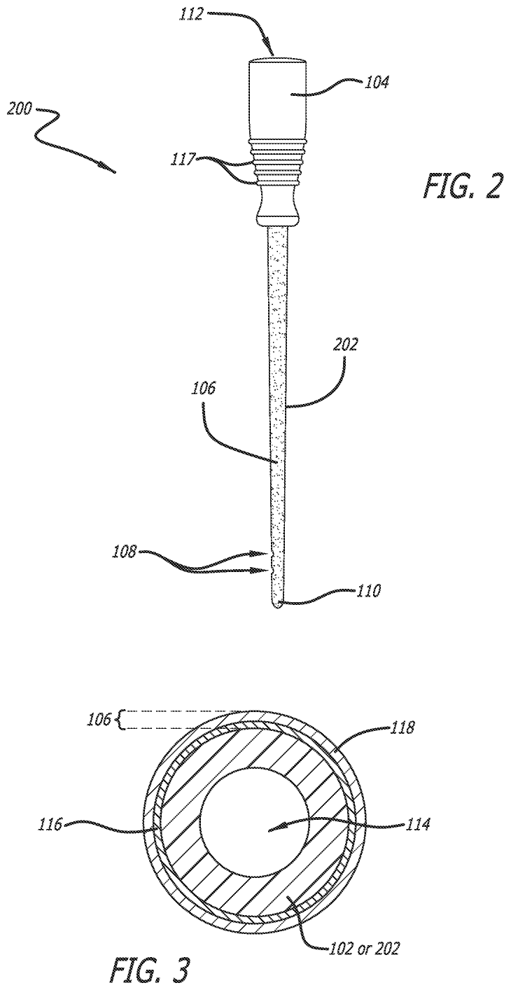

FIGS. 1 and 2 respectively illustrate a male urinary catheter 100 and a female urinary catheter 200 in accordance with some embodiments.

As shown, the urinary catheter 100 or 200 includes a catheter tube 102 or 202 and a funnel 104 coupled to a proximal portion of the catheter tube 102 or 202. The catheter tube 102 or 202 includes a lubricious coating 106 disposed over a surface of at least a distal portion of the catheter tube 102 or 202 up to an entirety of the catheter tube 102 or 202. Description for the catheter tube 102 or 202 and the funnel 104 are set forth immediately below; description for the lubricious coating 106 is set forth in the following section.

The catheter tube 102 and the catheter tube 202 differ in that the catheter tube 102 is longer than the catheter tube 202; however, each catheter tube of the catheter tubes 102 and 202 include a plurality of eyelets 108 about a catheter tip 110. The eyelets 108 are in fluid communication with the funnel opening 112 set forth below by way of a lumen 114 through the catheter. In addition, each catheter tube of the catheter tubes 102 and 202 including the surface thereof is of a polymeric material. The polymeric material is TPU, silicone, polyvinyl chloride, or a rubber. The rubber can be a natural rubber, a synthetic rubber, or a combination thereof such as red rubber, which includes a blend of natural rubber and styrene butadiene rubber. Optionally, the surface of the catheter tube 102 or 202 is modified by a plasma treatment to increase a surface energy SE of the polymeric material at the surface of the catheter tube 102 or 202, thereby promoting adhesion of the hydrophilic coating 116 set forth below.

The funnel 104 includes a funnel opening 112 that opens in a proximal end of the funnel 104 opposite the catheter tube 102 or 202 for voiding urine. The funnel opening 112 is in fluid communication with the eyelets 108 set forth above. In addition, the funnel 104 includes a plurality of ridges 117 integrated into an outer surface of the funnel 104. The ridges 117 are configured for gripping the funnel 104 as a handle while removing the urinary catheter 100 or 200 from the outer packaging 120 or 220 of the packaged urinary catheter 122 or 222 set forth below. Additionally or alternatively, the ridges 117 are configured for gripping the funnel 104 as a handle while inserting the urinary catheter 100 or 200 into a urethra.

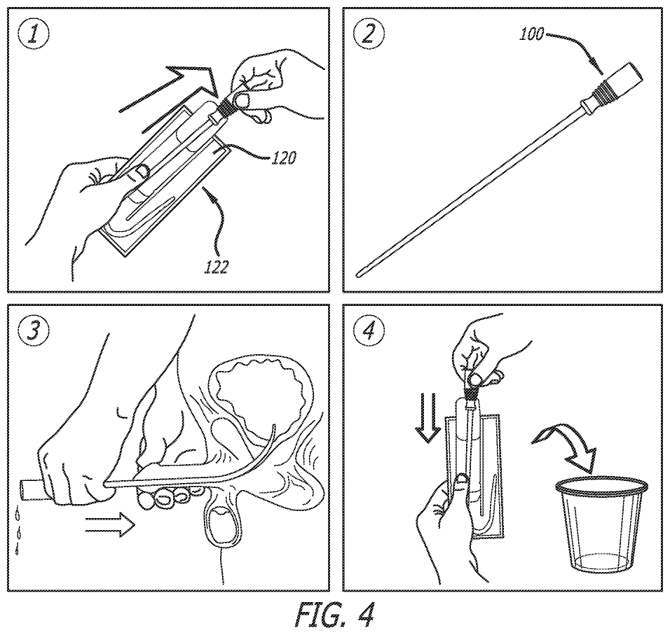

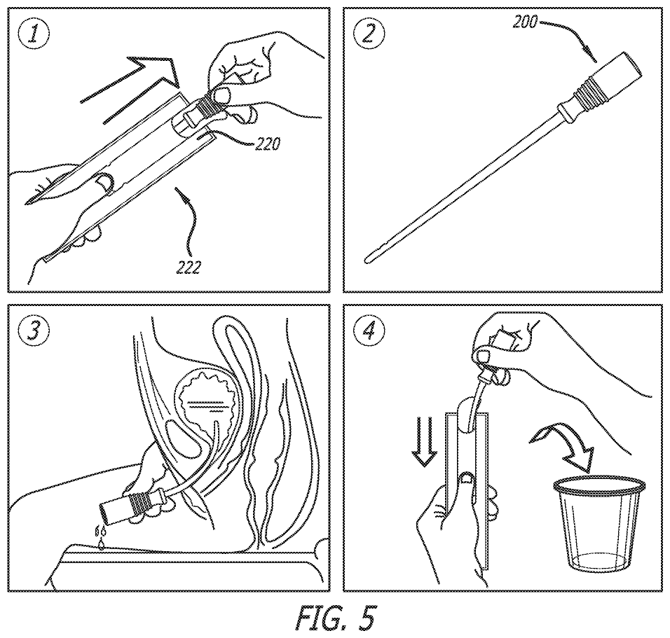

As shown in FIG. 4 or 4 the urinary catheter 100 or 200 can be disposed in an outer packaging 120 or 220 to form a packaged urinary catheter 122 or 222. Notably, the urinary catheter 100 or 200 disposed in the outer packaging 120 or 220 includes the lubricious coating 106 free of any water in addition to that of the lubricant 118 set forth below.

### Lubricious Coating

FIG. 3 illustrates a cross section of the male or female urinary catheter 100 or 200 in accordance with some embodiments.

Again, the lubricious coating 106 is disposed over the surface of at least the distal portion of the catheter tube 102 or 202 up to an entirety of the catheter tube 102 or 202. The lubricious coating 106 includes a hydrophilic coating 116 over the surface of the catheter tube 102 or 202 and a lubricant 118 over the hydrophilic coating 116.

The hydrophilic coating 116 can be a polyvinylpyrrolidone- or hyaluronic acid-based coating such as a hydrogel. Again, the surface of the catheter tube 102 or 202 can be modified by a plasma treatment to increase the surface energy SE of the polymeric material at the surface of the catheter tube 102 or 202, thereby promoting adhesion of the hydrophilic coating 116.

The lubricant 118 can be a combination of glycerin, water, glyceryl acrylate/acrylic acid copolymer, and propylene glycol. For example, the lubricant 118 can be 59.4-72.6% by weight of glycerin, 28.0-34.0% by weight of water, 1.58-1.93% by weight of glyceryl acrylate/acrylic acid copolymer, and 0.9-1.1% by weight of propylene glycol. Such a lubricant includes Lubrajel® CG by Ashland of Covington, KY.

Advantageously, the water of the lubricant 118 is at least partially absorbed by the hydrophilic coating 116 upon dipping the catheter tube 102 or 202 into the reservoir of the lubricant 118 as set forth below, thereby activating the hydrophilic coating 116. The glycerin, glyceryl acrylate/acrylic acid copolymer, and propylene glycol limit evaporation of any water from the lubricious coating 106 such that no water in addition to that of the lubricant 118 is needed for urethral insertion by urinary catheter users or their caregivers.

### Methods

Methods of the urinary catheter 100 or 200 include methods of making the urinary catheter 100 or 200. For example, a method of making the urinary catheter 100 or 200 can include one or more steps selected from a surface-treating step, a hydrophilic-coating applying step, a lubricant-dipping step, and a catheter-packaging step.

When performed, the surface-treating step includes treating the surface of at least the distal portion of the catheter tube 102 or 202 up to the entirety of the catheter tube 102 or 202 with a plasma treatment to modify the surface of the catheter tube 102 or 202 before performing the hydrophilic-coating step. As set forth above, the catheter tube 102 or 202 including the surface of the catheter tube 102 or 202 is of a polymeric material selected from TPU, silicone, polyvinyl chloride, and a rubber.

The hydrophilic coating-applying step includes applying the hydrophilic coating 116 over the surface of the catheter tube 102 or 202 of the urinary catheter 100 or 200. The hydrophilic coating-applying step includes allowing the hydrophilic coating 116 sufficient time to interact with one or more surface elements (e.g., pores, charged surface species from the surface-treating step, etc.) of the surface of the catheter tube 102 or 202 before performing the lubricant-dipping step.

The lubricant-dipping step includes dipping at least a distal portion of the catheter tube 102 or 202 into a reservoir of the lubricant 118 set forth above, thereby disposing the lubricant 118 over the hydrophilic coating 116. The lubricant-dipping step includes immersing the catheter tube 102 or 202 in the lubricant 118 with an immersion speed from about 0.15 in/s to about 0.50 in/s such as about 0.33 in/s. The lubricant-dipping step also includes withdrawing the catheter tube 102 or 202 from the lubricant 118 with a withdrawal speed from about 0.05 in/s to about 0.20 in/s such as about 0.12 in/s. Notably, The lubricant-dipping step includes substantially no dwell time in the lubricant 118 between the immersing the catheter tube 102 or 202 in the lubricant 118 and withdrawing the catheter tube 102 or 202 from the lubricant 118. And while the lubricant-dipping step can be repeated any number of times, a single dip in the lubricant 118 for the lubricant-dipping step provides sufficient lubricity for urethral insertion of the catheter tube 102 or 202.

The catheter-packaging step includes packaging the urinary catheter 100 or 200 in the outer packaging 120 or 220 without air drying the urinary catheter 100 or 200 after the lubricant-dipping step.

Methods of the urinary catheter 100 or 200 include methods of using the urinary catheter 100 or 200. For example, the method can include one or more steps selected from a catheter package-obtaining step, an unpackaging step, a catheter-inserting step, a urine-voiding step, a catheter-withdrawing step, a repackaging step, a resealing step, and a disposal step.

The catheter package-obtaining step includes obtaining the packaged urinary catheter 122 or 222 including the urinary catheter 100 or 200 disposed in the outer packaging 120 or 220. As set forth above, the catheter tube 102 or 202 of the urinary catheter 100 or 200 includes the lubricious coating 106 of the hydrophilic coating 116 over the surface of at least the distal portion of the catheter tube 102 or 202 and the lubricant 118 over the hydrophilic coating 116.

The unpackaging step includes removing the urinary catheter 100 or 200 from the outer packaging 120 or 220 by the funnel 104 of the urinary catheter 100 or 200.

The catheter-inserting step includes inserting the catheter tube 102 or 202 into a urethra. The lubricious coating 106 provides a user or his or her caregiver adequate time for successful urethral insertion of the catheter tube 102 or 202 by limiting evaporation of any water from the lubricious coating 106 form the unpackaging step through the catheter-inserting step that would otherwise cause tackiness.

The voiding step includes voiding urine from a bladder.

The catheter-withdrawing step includes withdrawing the catheter tube 102 or 202 from the urethra after the urine-voiding step.

The repackaging step includes placing the urinary catheter 100 or 200 in the outer packaging 120 or 220.

The resealing step includes resealing the urinary catheter 100 or 200 in the outer packaging 120 or 220. The resealing step prevents residual urine leakage from the urinary catheter 100 or 200.

The disposal step includes disposing of the urinary catheter 100 or 200.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. A urinary catheter, comprising:
a catheter tube of a polymeric material;
a lubricious coating over a surface of at least a distal portion of the catheter tube, the lubricious coating including a hydrophilic coating over the surface of the catheter tube and lubricant over the hydrophilic coating, wherein water of the lubricant is at least partially absorbed by the hydrophilic coating; and
a funnel coupled to a proximal portion of the catheter tube, the funnel including a funnel opening in a proximal end of the funnel for voiding urine.

2. The urinary catheter of claim 1, wherein the hydrophilic coating is a polyvinylpyrrolidone- or hyaluronic acid-based coating.

3. The urinary catheter of claim 1, wherein the lubricant is a combination of glycerin, water, glyceryl acrylate/acrylic acid copolymer, and propylene glycol.

4. The urinary catheter of claim 3, wherein the glycerin, glyceryl acrylate/acrylic acid copolymer, and propylene glycol limit evaporation of any water from the lubricious coating.

5. The urinary catheter of claim 1, wherein the polymeric material is thermoplastic polyurethane (“TPU”), silicone, polyvinyl chloride, or a rubber.

6. The urinary catheter of claim 1, wherein the surface of the catheter tube is a plasma-treated surface with a surface energy that promotes adhesion of the hydrophilic coating.

7. A method of making a urinary catheter, comprising:
applying a hydrophilic coating over a surface of a catheter tube of the urinary catheter; and
dipping at least a distal portion of the catheter tube into a reservoir of a lubricant, thereby disposing the lubricant over the hydrophilic coating, water of the lubricant at least partially absorbed by the hydrophilic coating.

8. The method of claim 7, wherein the dipping of the catheter tube into the reservoir of the lubricant includes immersing the catheter tube in the lubricant with an immersion speed from about 0.15 in/s to about 0.50 in/s.

9. The method of claim 7, wherein the dipping of the catheter tube into the reservoir of the lubricant includes immersing the catheter tube in the lubricant with an immersion speed of about 0.33 in/s.

10. The method of claim 7, wherein the dipping of the catheter tube into the reservoir of the lubricant includes withdrawing the catheter tube from the lubricant with a withdrawal speed from about 0.05 in/s to about 0.20 in/s.

11. The method of claim 7, wherein the dipping of the catheter tube into the reservoir of the lubricant includes withdrawing the catheter tube from the lubricant with a withdrawal speed of about 0.12 in/s.

12. The method of claim 7, wherein the dipping of the catheter tube into the reservoir of the lubricant includes substantially no dwell time in the lubricant.

13. The method of claim 7, wherein the dipping of the catheter tube into the reservoir of the lubricant includes a single dip in the lubricant.

14. The method of claim 7, wherein the lubricant is a combination of glycerin, water, glyceryl acrylate/acrylic acid copolymer, and propylene glycol.

15. The method of claim 14, wherein the glycerin, glyceryl acrylate/acrylic acid copolymer, and propylene glycol limit evaporation of any water from the lubricious coating.

16. The method of claim 7, further comprising packaging the urinary catheter without air drying the urinary catheter after the dipping of the catheter tube into the reservoir of the lubricant.

17. The method of claim 7, further comprising treating at least the distal portion of the catheter tube with a plasma treatment to modify the surface of the catheter tube before the applying of the hydrophilic coating over the surface of the catheter tube.

18. The method of claim 7, wherein the catheter tube is of a polymeric material selected from thermoplastic polyurethane (“TPU”), silicone, polyvinyl chloride, and a rubber.

19. The method of claim 7, wherein the hydrophilic coating is a polyvinylpyrrolidone- or hyaluronic acid-based coating.

20-23. (canceled)
